(19) United States
(12) Patent Application Publication
(10) Pub. No.: US 2024/0087729 A1
(43) Pub. Date: Mar. 14, 2024

(54) Outcome Matching with Personalized Comparison

(71) Applicant: Cercle.ai, Inc., Frisco, TX (US)

(72) Inventors: Juan Carlos RIVEIRO INSUA, Menlo Park, CA (US); Cristián MEROÑO GIL, Valencia (ES); David VEGAS GARCIA, Tulum (MX)

(21) Appl. No.: 18/235,030

(22) Filed: Aug. 17, 2023

Related U.S. Application Data
(60) Provisional application No. 63/398,815, filed on Aug. 17, 2022.

(51) Int. Cl. G16H 40/20

(52) U.S. Cl. CPC G16H 40/20

(57) ABSTRACT

A dynamic user interface is configured to generate two types of information that may be generated by sophisticated computer systems such as expert systems and/or artificial intelligence. These types and include a “what if” prediction that illustrates probable outcomes from alternative actions, and a “similarity” analysis which shows a user how adjustments to input data may affect the resulting predictions. The “what if” prediction provides users guidance as to what actions may be most beneficial. The “similarity” analysis helps the users understand consequences of medical decisions.

### CROSS-REFERENCE TO RELATED APPLICATIONS

This application claims priority to and benefit of US provisional patent application Ser. No. 63/398,815 filed Aug. 17, 2022. This application is related to U.S. provisional patent application Ser. No. 63/234,555 filed Aug. 18, 2021 and PCT application Ser. No. PCT/US22/40782 filed Aug. 18, 2022. The disclosures of all the above patent applications hereby incorporated herein by reference.

### BACKGROUND

### Field of the Invention

Embodiments of the invention are in the field of data presentation, and in some cases in the field of customized presentation of medical outcome predictions, such as IVF.

### Related Art

The black box nature of some artificial intelligence systems can hinder a user's understanding of and confidence in the generated results.

### SUMMARY

A dynamic user interface is configured to present to users both predictions of expected results that may occur from alternative actions and comparisons with similar sets of input data. The predictions are based on comparisons with a statistically relevant sets of reference data and may be made using statistical models and or trained neural networks. Comparisons with similar sets of input data are optionally configured to illustrate how variations in the input data can affect the probability of different outcomes. In the medical field, comparisons with similar sets of input data may include comparisons with patients having similar circumstances, e.g., similar medical profiles.

The dynamic user interface, thus, provides two types of information that are normally difficult to express in an informative manner, from the output of a sophisticated computer system. These types and include a “what if” prediction which illustrates probable outcomes from alternative actions, and a “similarity” analysis which shows a user how adjustments to input data may affect the resulting predictions. When the similarity analysis is made between two persons having similar profiles, this analysis may also serve to make the presented information more personal, in that a user can related to persons having similar profiles. This unique interface can serve to guide medical, and other types of decision making, and increase a user's understanding of the computer system's output.

For illustrative purposes, the dynamic user interface is discussed in relation to a healthcare information system, and specifically a system for supporting Invitro Fertilization (IVF). This healthcare information system may include logic elements and/or other hardware configured to generate, manage, and/or communicate the presented information. For example, the healthcare information system may include storage, interactive user interface logic, cohort identification logic, profile comparison logic, outcome prediction logic, and/or other logic discussed here.

As noted elsewhere herein, the decision support may be applied in other healthcare applications, and/or in other types of non-healthcare applications.

Various embodiments of the invention include; a data presentation system configured to present healthcare information to a patient, the system comprising: storage, including non-transient memory, configured to store patient medical histories and outcomes for a plurality of past patients; outcome prediction logic configured to predict a healthcare outcome for a patient based on one or more healthcare actions; profile comparison logic configured to compare the past patient medical histories to a medical history of the patient; a cohort identification logic configured to identify a cohort of members of the past patients having medical histories similar to the medical history of the patient; user interface logic configured to generate a user interface for presentation to the patient, the user interface including a representation of the predictions of healthcare outcomes generated by the outcome prediction logic, and also including interactive comparisons between the medical histories of the patient and medical histories of the cohort identified by the cohort identification logic; and a microprocessor configured to execute at least the user interface logic or the outcome prediction logic.

### BRIEF DESCRIPTION OF THE DRAWING

FIG. 1 illustrates a healthcare information system, according to various embodiments of the invention.

FIGS. 2-3 illustrate examples of dynamic user interfaces, which may be generated using the system illustrated in FIG. 1.

FIGS. 4A-4B illustrate expanded views of the dynamic user interfaces of FIGS. 2-3.

### DETAILED DESCRIPTION

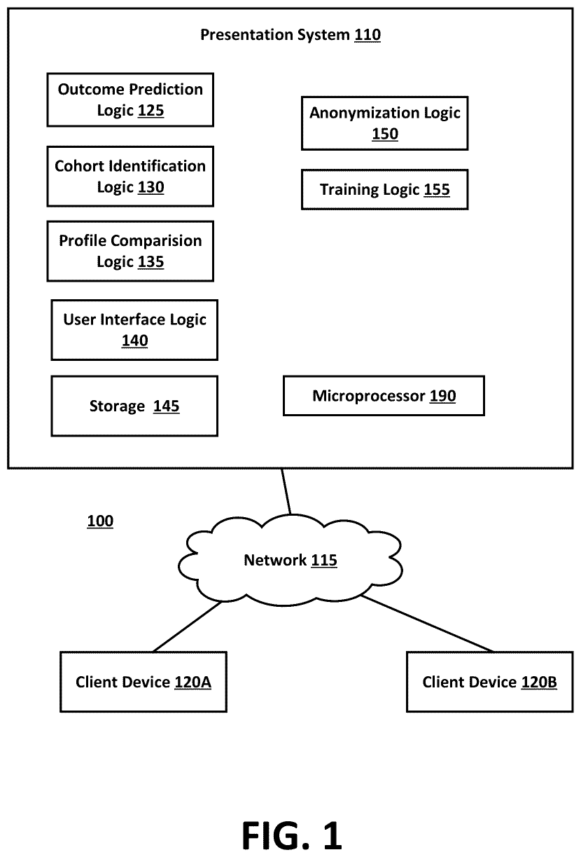

FIG. 1 illustrates a Healthcare Information System 100, according to various embodiments of the invention. Healthcare Information System 100 may be configured to, for example, present healthcare information to a patient and/or a healthcare provider. In various embodiments, Healthcare Information System 100, comprises a data Presentation System 110, a Network 115 and one or more Client Devices 120 (individually labeled, Client Device 120A, Client Device 120B, etc.). Presentation System 110 may be embodied on one or more computing devices. For example, on a plurality of web-based servers. Network 115 may include the internet, a private network, a local network and/or the like. Network 115 optionally includes wireless and/or wired communication channels. Client Devices 120 may include computing devices of patients, healthcare providers, administrations, and/or third parties. For example, Client Devices 120 may include a smartphone of a patient, a desktop computer of a physician, and/or the like. In some embodiments, Presentation System 110 is configured to communicate via Network 115 with an API to a medical records system.

Presentation System 110 includes a Storage 145 including non-transient memory. Storage 145 can include any type of computing memory including magnetic, optical, and/or electronic memory. Storage 145 is configured to store patient medical histories and outcomes for a plurality of past patients. Storage 145 may also be configured to store any combination of the logic discussed herein. For example, the various logic elements of Presentation System 110 or Client Devices 120 as taught herein.

Patient medical histories stored in Storage 145 optionally include: medical histories of a prospective birth mother, an egg donor, a sperm donor, and/or an embryo generated by fertilization of an egg from the egg donor. These histories can include any combination of medical data including outcomes, decisions made, treatments, pharmaceuticals administered, patient characteristics (e.g., age, weight, location, health history, race, genetic history, etc.). The histories can include data regarding current and/or past patients. For example, in some embodiments, histories of past patients are stored elsewhere and are used to train a machine learning system (e.g., Outcome Prediction Logic 125), while data concerning current patients are stored in Storage 145. Medical histories may further can include: reproductive histories, hormone levels, birth mother age, egg donor age, embryo growth rates, and/or embryo division times. Medical histories can include any of the medical information taught in U.S. provisional patent application Ser. No. 63/234,555.

In various embodiments, Storage 145 is configured to store a BioMedical graph such as that taught in U.S. provisional patent application Ser. No. 63/234,555 filed Aug. 18, 2021, and Outcome Prediction Logic 125 is configured to use the BioMedical graph to generate a prediction as taught therein.

Presentation System 110 further comprises Outcome Prediction Logic 125. Outcome Prediction Logic 125 is configured to predict a healthcare outcome for a (current) patient. In particular, Outcome Prediction Logic 125 is configured to predict invitro fertilization (IVF) outcomes. This prediction is based on one or more healthcare actions. For example, Outcome prediction Logic may be configured to predict an outcome for a particular patient if that patient performs a single embryo transfer and to predict an outcome for the particular patient if that patient performs a two embryo transfer. In various embodiments, Outcome Prediction Logic 125 is configured to predict invitro fertilization outcomes based on selection among alternative embryos, based on selection of a hormonal treatment, based on timing of embryo cell division, and/or based on morphology of an embryo. In various embodiments, Outcome Prediction Logic 125 includes any of the systems claimed in U.S. provisional patent application Ser. No. 63/234,555 filed Aug. 18, 2021.

Typically, Outcome Prediction Logic 125 is configured to provide comparable (quantitative) outputs. Specifically, Outcome Prediction Logic 125 is configured to make comparable predications based on alternative medical actions (e.g., treatments). For example, Outcome Prediction Logic 125 may be configured to predict healthcare outcomes based on alternative medical treatments and/or to provide a comparison between expected outcomes of the alternative medical treatments. In a specific example, Outcome Prediction Logic 125 may be configured to provide a quantitative prediction of serial embryo transfer and a quantitative prediction of parallel (multi-embryo) transfer. These to predictions being quantitative in that they can be numerically compared with each other, e.g., one predication gives a 25% chance of outcome “A” while another predictions gives a 45% chance of outcome “A.”

In various embodiments, Outcome Prediction Logic 125 is configured to predict outcomes based on multiple medical actions and to generate predictions based on various combinations of these medical actions. For example, a set of predictions may be generated based on the medical actions related to: number of eggs retrieved, type of embryo transfer and hormone supplements. And this set may include multiple predictions each based on a different combination of these actions, e.g., a prediction for 4 eggs retrieved plus serial embryo transfer and no provision of a specific hormone, a prediction for 8 eggs retrieved, multiple embryo transfer and provision of the specific hormone, and a prediction for 8 eggs retrieved plus serial embryo transfer and provision of the specific hormone at a specific time in the IVF cycle.

Presentation System 110 further includes Profile Comparison Logic 135. Profile Comparison Logic 135 is configured to compare past patient medical histories to a medical history of a current patient and to identify cohorts. Such comparison can include medical histories (events) and patient characteristics (e.g., age, number of births, etc.). In various embodiments, Profile Comparison Logic 135 is configured to 1) compare medical profiles in multiple dimensions, 2) generate an (optionally weighted) distance between medical profiles (cosign distance, Euclidean distance or any other type of distance between vectors) 3) generate a value representing profile distance based on a weighting of profile characteristics, and/or any combination thereof. For example, Profile Comparison Logic 135 may be configured to generate a cosign distance between medical profiles based on a weighting of different patient characteristics and/or medical events. In various embodiments, Profile Comparison Logic 135 is configured to provide a list of major differences between profiles, select/filter within specific ranges (age), weigh manageable differences differently than unmanageable differences, and/or the like. A manageable difference is one that can be changed, such as the weight or diet of a patient, an unmanageable difference is one that cannot be changed, such as a patients race or genetic history. In a specific example, Profile Comparison Logic 135 is optionally configured to make comparisons while distinguishing between static characteristics (filter by: age, race, past successful births, past miscarriages, etc.) and dynamic characteristics (hormone levels, weight, embryo incubation temperature, etc.). In various embodiments, Profile Comparison Logic 135 may be used to compare a current patient with specific historical patients or with aggregated historical patients.

Presentation System 110 optionally further includes Cohort Identification Logic 130. Cohort Identification Logic 130 is configured to identify one or more cohorts of members of the past patients having similar medical histories. The similarity may be weighted with regard to characteristics relevant to IFV outcomes. This identification may be used to categorize and/or group historical patients into cohorts. Cohort Identification Logic 130 may be configured to identify one or more cohorts of historical patients having a medical history and/or characteristics similar to those of a current patient.

Optionally, each cohort is distinguished by a different medical action taken among the historical patients. For example, Profile Comparison Logic 135 may be used to identify historical patients having similar medical profiles to a current patent (up to a point in fertility treatment). Cohort Identification Logic 130 may then be configured to identify a cohort of the identified historical patients have taken a medical action “B” and a cohort of the identified historical patients having taken a medical action “C”.

Thus, Cohort Identification Logic 130 and Profile Comparison Logic 135 may be used in at least two ways. In a first approach, Cohort Identification Logic 130 is used to divide historical patients into different cohorts and Profile Comparison Logic 135 is used to identify which of these cohorts are similar to a current patient. In a second approach, Profile Comparison Logic 135 is configured to identify historical patients similar to a current patient and Cohort Identification Logic 130 is then used to divide these identified historical patients into different cohorts. For example, the identified historical patients may be divided into cohorts based on medical treatment decisions they each made.

Cohort Identification Logic 130 optionally includes a trained machine learning system configured to separate patients in the cohorts discussed herein. Cohort Identification Logic 130 is optionally configured to perform statistical analysis on patient data in order to separate the patients into the cohorts discussed herein.

In an illustrative example, Profile Comparison Logic 135 and Cohort Identification Logic 130 may be used together as follows. Profile Comparison Logic 135 is first used to identify historical patients having medical profiles and characteristics similar to a current patient. Cohort Identification Logic 130 is then used to separate these identified historical patients into cohorts based on a medical treatment decision, e.g., what did the historical patents do next at the stage in their fertility treatments that was similar to a current stage of fertility treatment of the current patient. Each of these cohorts represents a different course of treatment open to the current patient. Each cohort may then be analyzed using Outcome Prediction Logic 125 to predict outcomes of specific treatment decisions.

The wealth of information provided by Outcome Prediction Logic 125, Profile Comparison Logic 135 and Cohort Identification Logic 130 is optionally conveyed to users, e.g., current patients and healthcare providers, using User Interface Logic 140. User Interface Logic 140 can include a variety of features that allow a user to interactively explore expected results of alternative medical actions. Such illustration and exploration produce important technical results. Specifically, the user can obtain a better understanding of the consequences of specific medical actions; complicated relationships between the patient's profile, medical decisions/actions and outcome probability is provided in a simplified understandable way; and a patient's confidence in calculated probability may be increased. These results lead directly to improvements in the medical decision-making process and allow users to increase the probability of successful IVF cycles, e.g., successful live births. The user interface(s) generated by User Interface Logic 140 are typically configured to be displayed on Client Devices 120. For example, within a browser or client application. The user interfaces are optionally HTML/XML based, and may be communicated securely using https.

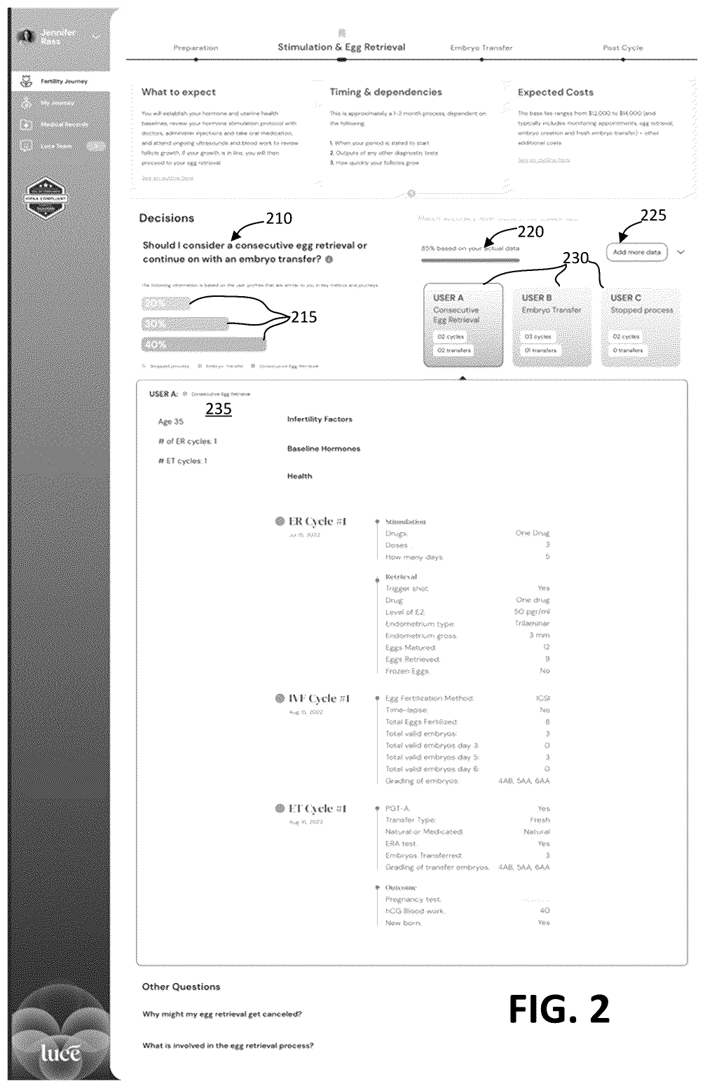

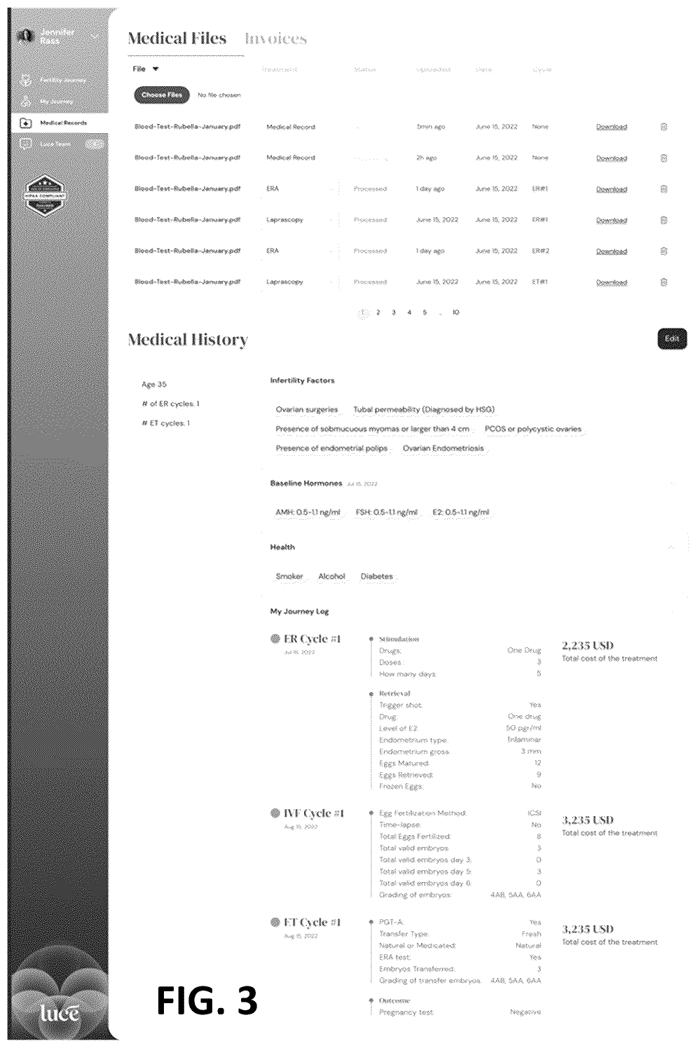

In various embodiments, the user interface generated by User Interface Logic 140 includes a representation of the predictions of healthcare outcomes generated by Outcome Prediction Logic 125, controls configured to make interactive comparisons between the medical histories of the current patient and medical histories of historical patients within a cohort, controls configured to show consequences of specific medical decisions, and/or other information and controls. FIGS. 2-3 illustrate examples of dynamic user interfaces, which may be generated using the system illustrated in FIG. 1, i.e., by User Interface Logic 140 based on data generated by Outcome Prediction Logic 125, Profile Comparison Logic 135, and/or Cohort Identification Logic 130. User Interface Logic 140 is optionally configured to generate a user interface for presentation to the patient, the user interface including a representation of the predictions of healthcare outcomes generated by the outcome prediction logic, and also including interactive comparisons between the medical histories of the current patient and medical histories of individual historical patients and/or a cohort of historical patients identified by the Cohort Identification Logic 130.

Referring specifically to FIG. 2, the particular User Interface 200 illustrates a “Decision to be Considered” 210. This information refers to the specific medical decision (choice of medical action) that is under consideration. The illustrated example presents the treatment question “Should I consider a consecutive egg retrieval or continue on with an embryo transfer?”

Percentages 215 of decisions made by a group of historical patents having similarity to the current patient, e.g., made in a cohort of historical patients similar to the current patient, are also illustrated in User Interface 200. The exemplary values indicate that 20% of historical patients stopped treatment, 30% of historical patients chose embryo transfer, and 40% of patients chose consecutive egg retrieval.

User Interface 200 further includes a Data Match 220 between the current patient and either specific historical users or a cohort thereof. This value is indicative of how close a specific historical patient or cohort thereof matches the current patient. Optionally, User Interface Logic 140 is configured such that clicking on Data Match 220 produces a list of the greatest differences and/or similarities between the current patient and the specific historical patient or cohort thereof. The value included in Data Match 220 is optionally generated by Profile Comparison Logic 135 as described elsewhere herein. In various embodiments, a user can select the weighting of data used in the comparison, filter which data is compared, and/or otherwise choose how the value is calculated.

User Interface 200 further includes an Add Data option 225 configured to add information to the user's profile. In some embodiments, selecting this option results in the interface illustrated in FIG. 3. This interface allows a user to upload medical files and/or edit their personal characteristics. In some embodiments, the interface illustrated in FIG. 3 includes indications of user characteristics under the control of the user, e.g., a smoking habit or consumption of alcohol, which may improve IVF outcome if changed. The user can vary these characteristics and examine how they impact IVF (or other medical) outcomes. In some embodiments, User Interface Logic 140 is configured to suggest changes in characteristics of the current patient that are under the control of the current patient, and in particular those changes that are most likely to improve outcomes. In some embodiments the interface illustrated in FIG. 3 is configured to suggest which additional data would be desirable to add to a current patient's profile. Medical records added to the patient's profile are optionally processed using a machine learning system configured to extract relevant information from the medical records. The uploaded record may include images, narrative healthcare provider notes, laboratory results, information from an electronic medical records system, and/or the like.

User Interface 200 further includes Representations 230 of two or more medical actions selected by historical patients. In the exemplary illustration of FIGS. 2, three actions are illustrate as represented by “User A,” “User B” and “User C”. These “users” may be actual historical users or representative aggregations (cohorts) thereof. Optionally, selecting one of the Representations 230 results in further detail regarding that representation including, for example, outcome probabilities for that medical action, individual historical patient profiles, comments from patients added to their profiles, comments from healthcare providers added to their profiles, costs, and/or the like. In some embodiments, a user may scroll or use controls to see additional Representations 230. For example, by clicking on one of the Percentages 215, User Interface Logic 140 may be configured to show Representations 230 of users that took as specific medical action. Further, by selecting a specific Representations 230, illustrative Patent Profiles 235 of the historical user (or cohort thereof) may be displayed. These displayed profiles may include highlights of differences between the displayed profile and the profile of the current user. When displayed to other users, all medical records and profiles are anonymous, no personal information is included.

Presentation System 110 optionally further includes Anonymization Logic 150. Anonymization Logic 150 is configured to anonymize the past patient medical histories. Anonymization is intended to remove information that may be used to identify a specific patient.

Presentation System 110 optionally further includes Training Logic 155. Training Logic 115 is configured to train machine learning system, which made be included in Outcome Prediction Logic 125, Cohort Identification Logic 130, and/or Profile Comparison Logic 135. For example, Training Logic 115 may be configured to train a neural network using historical patient data (e.g., medical histories including outcomes and/or patient characteristics). In some embodiments, Training Logic is configured to train a machine learning system based on data of a specific cohort of historical patients.

Presentation System 110 includes at least one Microprocessor 190. Microprocessor 190 is configured to execute any one or more of the various logic elements illustrated in FIG. 1. Microprocessor 190 may include, for example, a digital microprocessor configured to execute computing instructions. Microprocessor 190 can include electronic, optical, and/or quantum components. Presentation System 110 optionally includes a plurality of Microprocessor 190 distributed over multiple computing devices.

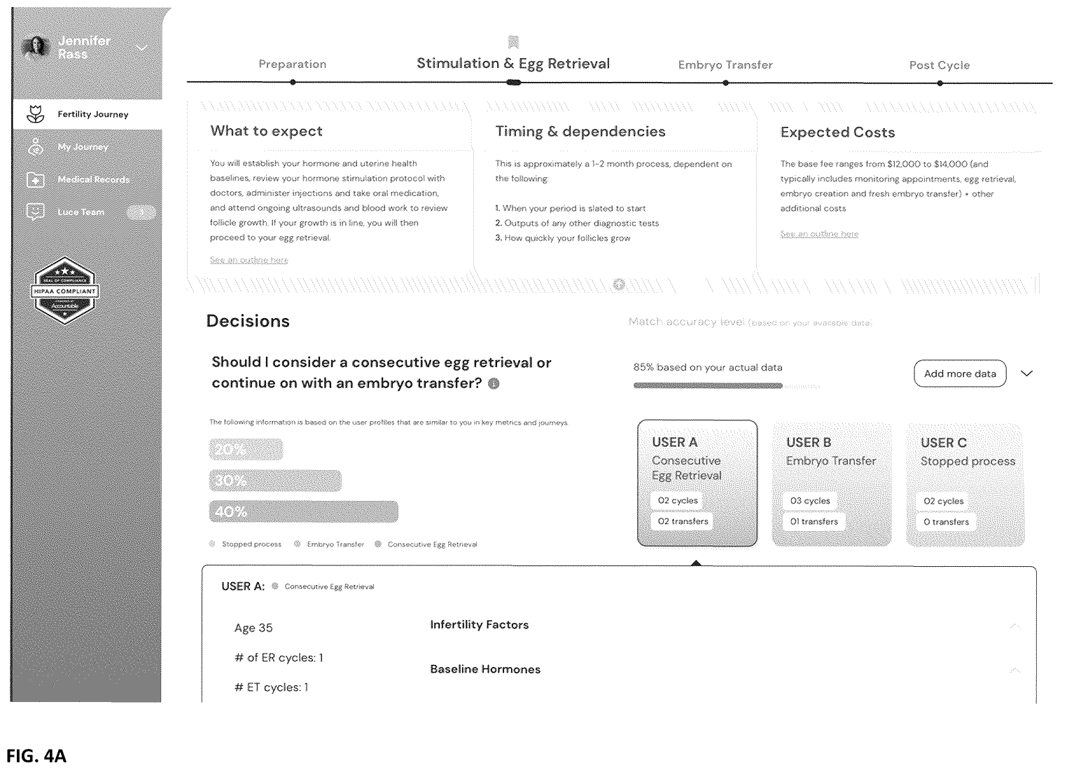

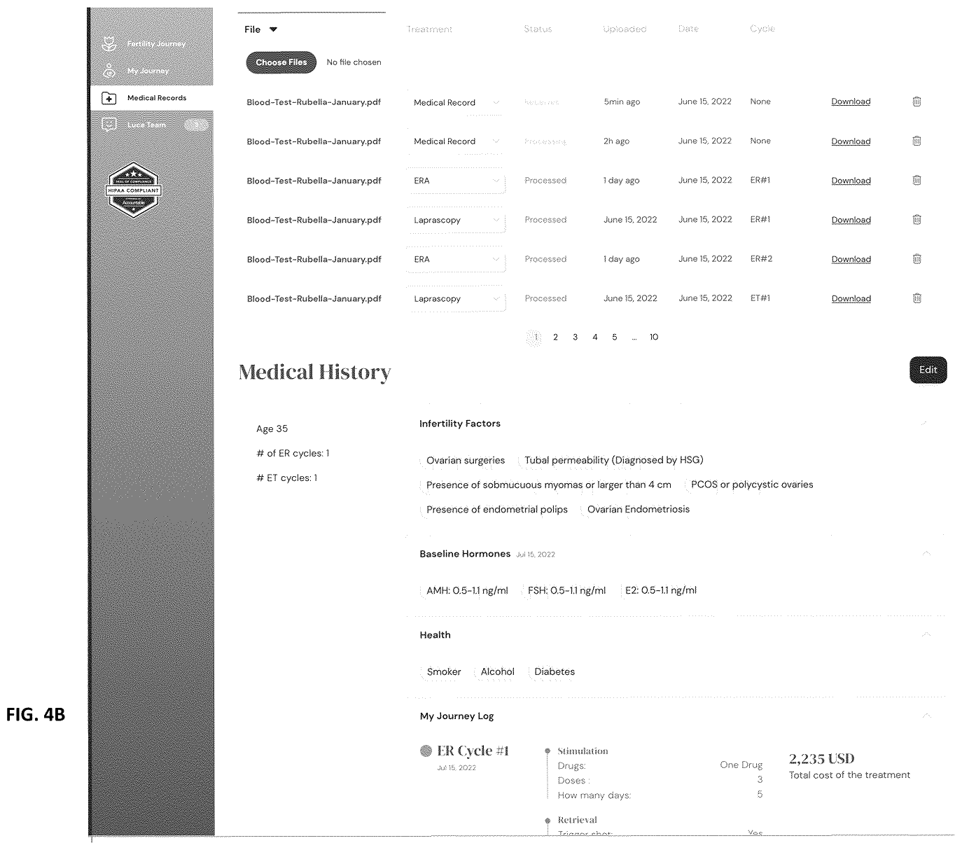

FIGS. 4A-4B illustrate expanded views of the dynamic user interfaces of FIGS. 2-3.

Several embodiments are specifically illustrated and/or described herein. However, it will be appreciated that modifications and variations are covered by the above teachings and within the scope of the appended claims without departing from the spirit and intended scope thereof. For example, the described systems and methods may be applied to other medical situations or decision making processes. For example, the systems and methods may be adapted to medical situations involving: Mental health, Oncology/Cancer, Alzheimer, Parkinson, autoimmune diseases, complex surgery evaluation to assess the risk versus the benefit, heart problems, injuries when you combine them with wearables to have early prediction of health problems or when combine with DNA, ultra personalization for almost every treatment in terms of dose and drugs, and/or the like. The systems and methods described herein may also be applied to non-medical decision making including, for example, for Career planning, college applications, investments, buying houses, selecting school for kids, best nutrition or diet.

The embodiments discussed herein are illustrative of the present invention. As these embodiments of the present invention are described with reference to illustrations, various modifications or adaptations of the methods and or specific structures described may become apparent to those skilled in the art. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

Computing systems and/or logic referred to herein can comprise an integrated circuit, a microprocessor, a personal computer, a server, a distributed computing system, a communication device, a network device, or the like, and various combinations of the same. A computing system or logic may also comprise volatile and/or non-volatile memory such as random access memory (RAM), dynamic random access memory (DRAM), static random access memory (SRAM), magnetic media, optical media, nano-media, a hard drive, a compact disk, a digital versatile disc (DVD), optical circuits, and/or other devices configured for storing analog or digital information, such as in a database. A computer-readable medium, as used herein, expressly excludes paper. Computer-implemented steps of the methods noted herein can comprise a set of instructions stored on a computer-readable medium that when executed cause the computing system to perform the steps. A computing system programmed to perform particular functions pursuant to instructions from program software is a special purpose computing system for performing those particular functions. Data that is manipulated by a special purpose computing system while performing those particular functions is at least electronically saved in buffers of the computing system, physically changing the special purpose computing system from one state to the next with each change to the stored data.

The “logic” discussed herein is explicitly defined to include hardware, firmware or software stored on a non-transient computer readable medium, or any combinations thereof. This logic may be implemented in an electronic and/or digital device (e.g., a circuit) to produce a special purpose computing system. Any of the systems discussed herein optionally include a microprocessor, including electronic and/or optical circuits, configured to execute any combination of the logic discussed herein. The methods discussed herein optionally include execution of the logic by said microprocessor.

What is claimed is:

1. A data presentation system configured to present healthcare information to a patient, the system comprising:
storage, including non-transient memory, configured to store patient medical histories and outcomes for a plurality of past patients;
outcome prediction logic configured to predict a healthcare outcome for a current patient based on one or more healthcare actions;
profile comparison logic configured to compare the past patient medical histories to a medical history of the patient;
a cohort identification logic configured to identify a cohort of members of the past patients having medical histories similar to the medical history of the patient;
user interface logic configured to generate a user interface for presentation to the patient, the user interface including a representation of the predictions of healthcare outcomes generated by the outcome prediction logic, and also including interactive comparisons between the medical histories of the patient and medical histories of the cohort identified by the cohort identification logic; and
a microprocessor configured to execute at least the user interface logic or the outcome prediction logic.

2. The system of claim 1, wherein the outcome prediction logic is configured to predict invitro fertilization (IVF) outcomes.

3. The system of claim 2, wherein the outcome prediction logic is configured to predict invitro fertilization outcomes based on selection among alternative embryos, based on selection of a hormonal treatment, based on timing of embryo cell division, or based on morphology of an embryo.

4. The system of claim 2, wherein the outcome prediction logic is configured to predict healthcare outcomes based on alternative medical treatments or to provide a comparison between expected outcomes of the alternative medical treatments.

5. The system of claim 2, wherein the user interface is configured for comparison of a profile of a current patient to multiple profiles of historical patients or cohorts thereof.

6. The system of claim 2, wherein the user interface is configured to wherein the profile of the current patient is compared to profiles of similar historical patients, as determine by the profile comparison logic.

7. The system of claim 2, wherein the user interface is configured to display percentages of historical patients that chose different medical actions at a particular point in IVF treatment.

8. The system of claim 2, wherein the user interface is configured to show a similarity between a current patient and an historical patient or a cohort thereof.

9. The system of claim 2, wherein the user interface is configured to

10. The system of claim 2, wherein the user interface includes a map of decisions or similar patient profiles, and is configured for a current patient to navigate this map to explore predicted outcomes following different medical actions.

11. The system of claim 2, wherein the profile comparison logic is configured for a user to select between comparisons of their profile to a cohort of historical users or comparisons of their profile to individual historical users.

12. The system of claim 2, wherein the profile comparison logic is configured to 1) compare medical profiles in multiple dimensions, 2) generate a Cosine distance between medical profiles, 3) generate a value representing profile distance based on a weighting of profile characteristics.

13. The system of claim 2, wherein the profile comparison logic is configured to distinguish between static characteristics and dynamic characteristics, the dynamic characteristics including hormone levels, patient weight, or embryo incubation temperature).

14. The system of claim 2, wherein the cohort identification logic is configured to identify the cohort of members of the past patents having medical histories similar to the medical history of the patient, the similarity being weighted with regard to characteristics relevant to IFV outcomes.

15. The system of claim 2, wherein the patient medical histories include: medical histories of a prospective birth mother, an egg donor, and/or an embryo generated by fertilization of an egg from the egg donor.

16. The system of claim 2, wherein the patient medical histories include: reproductive histories, hormone levels, birth mother age, egg donor age, embryo growth rates, and/or embryo division times.

17. The system of claim 2, wherein the storage is further configured to store a graph, and the outcome prediction logic is configured to use the graph to generate a prediction.

18. The system of claim 2, further comprising anonymization logic configured to anonymize the past patient medical histories.
